(19) United States
(12) Patent Application Publication
(10) Pub. No.: US 2013/0202688 A1
(43) Pub. Date: Aug. 8, 2013

(54) DELAYED RELEASE ORAL DISINTEGRATING PHARMACEUTICAL COMPOSITIONS OF LANSOPRAZOLE

(71) Applicants: Sunilendu Bhushan Roy, Gujarat (IN); Sushrut Krishnaji Kulkarni, Gujarat (IN); Hemant Manilal Mamania, Gujarat (IN)

(72) Inventors: Sunilendu Bhushan Roy, Gujarat (IN); Sushrut Krishnaji Kulkarni, Gujarat (IN); Hemant Manilal Mamania, Gujarat (IN)

(21) Appl. No.: 13/695,646

(22) PCT Filed: May 3, 2011
(86) PCT No.: PCT/IN2011/000308

§ 371 Date: Dec. 19, 2012

(30) Foreign Application Priority Data
May 4, 2010 (IN) 1416/MUM/2010

(51) Int. Cl. A61K 9/14

(52) U.S. Cl. CPC A61K 9/14
USPC 424/451; 424/464; 424/490; 424/494; 424/497; 514/338

(57) ABSTRACT

The present invention relates to delayed release oral disintegrating pharmaceutical compositions of lansoprazole or pharmaceutically acceptable salts thereof. The invention also relates to processes for the preparation of such compositions.

### FIELD OF THE INVENTION

The present invention relates to delayed release oral disintegrating pharmaceutical compositions of lansoprazole or pharmaceutically acceptable salts thereof. The invention also relates to processes for the preparation of such compositions.

### BACKGROUND OF THE INVENTION

Lansoprazole is a strong inhibitor of proton pump and is widely used as a therapeutic agent for stomach ulcer, duodenal ulcer, and gastro esophageal reflux disorders since it effectively inhibits gastric acid secretion.

Lansoprazole is disclosed in U.S. Pat. Nos. 4,628,098 and 4,689,333. Chemically, it is 2-[[[3-methyl-4-(2,2,2-trifluoroethoxy)-2-pyridyl]methyl]sulfinyl]benzimidazole, having the following structural formula:

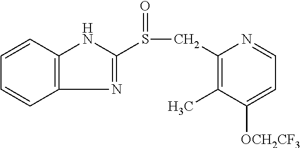

Because of the strong tendency of lansoprazole to decompose in a neutral and in particular, acidic environment, numerous approaches have been tried to form a stable pharmaceutical formulation comprising lansoprazole. In addition, stability of lansoprazole in the form of dosage forms, i.e., tablets, powders, fine granules, capsules, etc. is compromised due to possibility of strong interaction between the drug and other components in the composition.

The delayed release orally disintegrating tablets of lansoprazole are currently being marketed in the US under the name Prevacid® by Takeda in the strengths of 15 mg and 30 mg.

U.S. Pat. No. 5,626,875 discloses a pharmaceutical formulation of substituted 2-(2-pyridylmethyl)sulfinyl-1H-benzimidazole compound that is devoid of an alkaline stabilizer. Instead, a non-alkaline isolation layer is used to separate the core containing benzimidazole compounds from the acidic enteric coat.

U.S. Pat. No. 5,464,632 discloses a rapidly disintegrable multiparticulate tablet, the excipient mixture of which is suitable for imparting a disintegration rate such that the tablet disintegrates in the mouth in less than sixty seconds, characterized by the fact that the active substance is present in the form of coated microcrystals or coated or uncoated microgranules.

U.S. Pat. No. 6,328,994 discloses an orally disintegrable lansoprazole tablet which comprises fine enteric coated granules having an average particle diameter of 400 μm or less.

European Patent No. EP 0723437 B1 discloses an oral pharmaceutical multiple unit tableted dosage form, comprising individually enteric coating layered units characterized in that the enteric coating layer has a particular thickness, and comprises a plasticizer in an amount of 15 to 50% by weight of the enteric coating layer polymer.

U.S. Pat. No. 6,706,285 discloses an enteric coated lansoprazole having a core and a film of an enteric coating agent on the surface thereof, wherein the core contains a complex of the lansoprazole and an ion-exchange resin.

U.S. Pat. Nos. 4,786,505 and 4,853,230 disclose compositions of benzimidazole which are stabilized by the use of an alkaline reacting compound in the core. The compositions also contain a protective sub-coating and an enteric outer coating. The separating layer is made up of water-soluble polymeric substances, which separates the alkaline core from the acidic enteric coating.

U.S. Patent Application No. 20060165781 discloses orally disintegrating tablets containing active ingredients with specific particle size distribution, spray dried mannitol, microcrystalline cellulose of specific particle size distribution and sodium croscarmellose.

The composition is prepared by direct compression.

Although various attempts have been made in the prior art to develop stable formulations containing benzimidazole compounds, such as lansoprazole, but it was generally believed that lansoprazole composition containing enteric coated granules having average particle size more than 400 μm results in roughness or oral discomfort.
In the present invention we have now found that pharmaceutical compositions comprising lansoprazole, when prepared with enteric coated granules having average particle size more than 400 μm, are stable and the composition disintegrates in less than 30 seconds in water at 37° C.

### SUMMARY OF THE INVENTION

In one general aspect there is provided an orally disintegrating pharmaceutical composition that includes enteric coated granules of lansoprazole or pharmaceutically acceptable salts thereof, wherein the enteric coated granules have an average particle diameter of more than 400 μm.

In another general aspect there is provided an orally disintegrating pharmaceutical composition that includes enteric coated granules having an average particle diameter of more than 400 μm, wherein the enteric coated granules include: (a) a drug core comprising lansoprazole or pharmaceutically acceptable salts thereof, one or more pharmaceutically acceptable excipients, and optionally one or more alkalizing agents;

(b) an optional barrier coating layer over the drug core comprising one or more pharmaceutically acceptable excipients, and optionally one or more alkalizing agents; and
(c) an outer enteric coating layer comprising one or more enteric polymers, optionally with one or more pharmaceutically acceptable controlled release polymers.

Embodiments of the present invention may include one or more of the following features. For example, the pharmaceutical composition may further include one or more pharmaceutical acceptable excipients. The pharmaceutical acceptable excipients may include diluents, disintegrants, plasticizers, alkalizing agents, binder, glidants, sweeteners, buffering agents, fillers and lubricants.

In another general aspect there is provided a process for the preparation of an orally disintegrating pharmaceutical composition of lansoprazole or pharmaceutically acceptable salts thereof. The process includes providing enteric coated granules comprising lansoprazole or pharmaceutically acceptable salts thereof, one or more pharmaceutically acceptable excipients, and optionally one or more alkalizing agents, wherein the enteric coated granules have an average particle diameter of more than 400 μm; forming a mixture by mixing the enteric coated granules with one or more pharmaceutically acceptable excipients; and forming the mixture into a pharmaceutical dosage form.

In another general aspect there is provided a process for the preparation of an orally disintegrating pharmaceutical composition of lansoprazole or pharmaceutically acceptable salts thereof that includes enteric coated granules of lansoprazole or pharmaceutically acceptable salt thereof. The process for the preparation of the enteric coated granules includes the steps of:

(a) preparing granules comprising lansoprazole or pharmaceutically acceptable salt thereof, one or more pharmaceutically acceptable excipients, and optionally one or more alkalizing agents;
(b) optionally, providing a barrier coating layer over the granules comprising one or more pharmaceutically acceptable excipients, and optionally one or more alkalizers; and
(c) providing an enteric coating layer over the granules of step (a) or barrier coating layer to prepare the enteric coated granules,
wherein the enteric coated granules have an average particle diameter of more than 400 μm.

In another general aspect there is provided an orally disintegrating pharmaceutical composition that includes enteric coated granules of lansoprazole or pharmaceutically acceptable salts thereof, the enteric coated granules having an average particle diameter of more than 400 μm, wherein the composition disintegrates in less than 30 seconds in water at 37° C.

In another general aspect, there is provided an orally disintegrating pharmaceutical composition that includes enteric coated granules of lansoprazole or pharmaceutically acceptable salts thereof, the enteric coated granules having an average particle diameter of more than 400 μm, wherein the enteric coating is about 35% to about 55% based on total weight of the enteric coated granules in the composition.

In another general aspect there is provided an orally disintegrating pharmaceutical composition that includes enteric coated granules of lansoprazole or pharmaceutically acceptable salts thereof, the enteric coated granules having an average particle diameter of more than 400 μm, wherein the composition exhibits no significant difference in rate and or extent of absorption of lansoprazole or pharmaceutically acceptable salts thereof as compared to orally disintegrating formulation commercially marketed under the trade name Prevacid®.

In another general aspect of the invention, there is provided a stable orally disintegrating pharmaceutical composition that includes enteric coated granules of lansoprazole or pharmaceutically acceptable salts thereof, the enteric coated granules having an average particle diameter of more than 400 μm, wherein the composition retains at least 80% of the potency of lansoprazole or pharmaceutically acceptable salts thereof in the pharmaceutical composition after storage for three months at 40° C. and 75% relative humidity.

Embodiments of the present invention may include one or more of the following features for example the pharmaceutical composition may further include one or more pharmaceutical acceptable excipients. The pharmaceutical acceptable excipients may include diluents, disintegrants, plasticizers, alkalizing agents, binder, glidants, sweeteners, buffering agents, fillers and lubricants.

In another general aspect of the invention there is provided a method of treating gastroesophageal reflux disease or a symptom thereof in a subject in need thereof, the method comprising administering an orally disintegrating pharmaceutical composition that include enteric coated granules of lansoprazole or pharmaceutically acceptable salts thereof, wherein the enteric coated granules having an average particle diameter of more than 400 μm.

The details of one or more embodiments of the present invention are set forth in the description below. Other features, objects and advantages of the invention will be apparent from the description.

### DETAILED DESCRIPTION OF THE INVENTION

The inventors of the present invention have surprisingly found that even when lansoprazole is formulated into an orally disintegrating pharmaceutical composition in the form of enteric coated granules having an average particle diameter of more than 400 μm; it does not exhibit any incompliance in terms of roughness or oral discomfort. Moreover, such composition also exhibit excellent stability upon storage and is bioequivalent when compared with marketed formulation, Prevacid®.

The formulations of the present invention are stable and may retain at least 80% of the potency of lansoprazole or pharmaceutically acceptable salts thereof in the pharmaceutical composition after storage for three months at 40° C. and 75% relative humidity.

Further, inventors of the present invention have found that the granules in the composition of the invention when coated with about 35% to about 55% enteric coating layer based on total weight of the enteric coated granules, the resulting composition may remain stable over the storage period.

Embodiments of the present invention relate to orally disintegrating pharmaceutical compositions of lansoprazole or pharmaceutically acceptable salts thereof that include enteric coated granules, wherein the enteric coated granules have an average particle diameter of more than 400 μm.

The term ‘lansoprazole’ used throughout the specification refers to lansoprazole, its pharmaceutically acceptable salts, pharmaceutically acceptable solvates, pharmaceutically acceptable hydrates, pharmaceutically acceptable enantiomers, pharmaceutically acceptable derivatives, pharmaceutically acceptable polymorphs and pharmaceutically acceptable prodrugs thereof.

The term ‘alkalizing agent’ indicates an agent capable of imparting stability to the active agent present in the composition. Suitable alkalizing agents may include one or more of sodium aluminium silicate, calcium silicate, magnesium aluminometasilicate, magnesium aluminosilicate, magnesium aluminum silicate, magnesium aluminate, dry aluminum hydroxide, synthetic hydrotalcite, synthetic aluminum silicate, magnesium carbonate, calcium carbonate, magnesium oxide, aluminum hydroxide, sodium hydrogencarbonate, L-arginine, sodium phosphate, disodium hydrogenphosphate, sodium dihydrogenphosphate, potassium phosphate, dipotassium hydrogenphosphate, potassium dihydrogenphosphate, disodium citrate, sodium succinate, ammonium chloride, sodium benzoate, sodium carbonate, potassium carbonate, sodium bicarbonate, potassium hydrogen carbonate, sodium phosphate, potassium phosphate, calcium dibasic phosphate, trisodium phosphate, sodium hydroxide, potassium hydroxide, lithium hydroxide, meglumine, and the like.

The alkalizing agent can be present in the composition in an amount sufficient to render stability to the drug in the acidic environment. The alkalizing agent may be present in drug core and barrier coat layer, respectively in amount ranging from 1% to about 15% and about 0.01% to about 2% by total weight of the composition.

The term ‘granule’ can include granules or pellets or tablets or minitablets or particles or microparticles.

The granules can be prepared either by dry granulation, wet granulation, or direct compression of lansoprazole or pharmaceutically acceptable salts thereof with one or more pharmaceutically acceptable excipients, or layering lansoprazole or pharmaceutically acceptable salts thereof on inert core or by extrusion/spheronization process.

The term ‘orally disintegrating composition’ can include composition which disintegrates within 60 seconds, for example within 30 seconds in water at 37° C.

The enteric coated granules of the present invention have an average particle diameter of more than 400 μm. In particular, the average particle diameter of the enteric coated granules is about 400 μm to about 750 μm. For example, the average particle diameter of the granules is about 420 μm to about 710 μm.

The enteric coated granules can be further coated with a finishing coat, film coat or over-coating layer and compressed into tablets. For example, the enteric coated granules are further coated with one or more over-coating layers of sugars or sugar alcohols such as sorbitol, lactitol, mannitol, maltitol etc. The amount of over-coating layer in the composition preferably ranges from about 2% to about 8% by total weight of the composition.

The orally disintegrating pharmaceutical composition of the present invention can be formulated into tablets, minitablets, pellets, granules, capsules, chewables, pellets in capsule, granules in capsule, minitablets in capsule, granules in sachet or any other dosage form suitable for oral administration.

In general, the orally disintegrating pharmaceutical composition of the present invention may be in the form of enteric coated pellets or granules. Alternatively, the pellets or granules can be processed further into solid dosage forms such as tablet, minitablets or said multiple units can be filled into capsules or sachets.

In a further embodiment, the orally disintegrating pharmaceutical composition may be in the form of enteric coated granules comprising a core of lansoprazole or pharmaceutically acceptable salts thereof, the core optionally comprising one or more alkalizing agent; optionally a barrier coating layer on the said core; and an outer enteric coating layer comprising enteric polymer(s) which may further comprise one or more pharmaceutically acceptable controlled release polymers. The amount of barrier coating layer in the composition may range from about 5% to about 15% by total weight of the composition.

In a further embodiment, the core of lansoprazole or pharmaceutically acceptable salts thereof may be in the form of inert cores coated with lansoprazole or pharmaceutically acceptable salts thereof or granules or pellets of lansoprazole or pharmaceutically acceptable salts thereof comprising one or more pharmaceutically acceptable excipients prepared using methods known to person skilled in the art, such as dry granulation, wet granulation, spray drying, extrusion-spheronization, hot melt extrusion.

The amount of drug cores present in the composition may range from about 35% to about 65% by total weight of the composition.

In a further embodiment, the orally disintegrating pharmaceutical composition in the form of a tablet, comprising (i) enteric coated granules having an average particle diameter of more than 400 μm, which enteric coated granules comprise: (a) a core comprising lansoprazole or pharmaceutically acceptable salts thereof, one or more pharmaceutically acceptable excipients, and an alkalizing agent; (b) a barrier coating layer comprising one or more pharmaceutically acceptable excipients and an alkalizing agent; (c) an enteric coating layer over the barrier coating layer comprising one or more enteric coating polymer(s) and optionally one or more pharmaceutically acceptable controlled release polymer(s); and (c) an outer over-coating layer comprising sugar alcohol over the enteric coating layer; wherein the amount of said enteric coating layer is about 35% to about 55% based on total weight of the enteric coated granules.

The inert core can be made of inert non-pareil sugar spheres, microcrystalline cellulose, mannitol, lactose beads and the like. Both drug layer and barrier coating layer may comprise one or more alkaline agent/s.

The inert core may be a microcrystalline sphere. The inert core may have a diameter of about 150 μm to about 400 μm, for example between 150 μm to 300 μm.

Enteric coated compositions are those which are coated with enteric polymer/s. Suitable “enteric polymer/s” may include one or more of hydroxypropylmethyl cellulose phthalate, cellulose acetate phthalate, polyvinyl acetate phthalate, methyl cellulose phthalate, copolymerized methacrylic acid/methacrylic acid methyl esters, methacrylate copolymer [e.g., commercially available grades of Eudragit such as S 12.5P, S 12.5, S 100, FS 30D, L 12.5P, L12.5, L 100, L 100-55 and L30D-55 etc] Kollicoat MAE30DP, carboxymethylcellulose, shellac, etc. The amount of enteric coating layer in the composition preferably ranges from about 30% to about 60% by total weight of composition.

The enteric coating layer may contain plasticizers such as triethyl citrate, polyethylene glycol, acetylated monoglyceride, triacetin, castor oil, or mixtures thereof and is usually in the range of 1 to 50% by weight of the enteric coating or enteric coating layer polymer/s. Preferably, the enteric coating layer comprises one or more enteric polymer/s and pharmaceutically acceptable controlled release polymer/s respectively in the ranging from about 15% to about 30% and about 0.5% to about 5% by total weight of the composition.

The enteric coating layer(s) constitutes a thickness of approximately at least 10 μm, preferably more than 20 μm.

Pharmaceutically acceptable controlled release polymers may include hydrophilic and/or hydrophobic controlled release polymers known in the art. Other controlled release providing substances such as fats, lipids, waxes may also be used. Preferably, controlled release polymer is hydrophobic polymer selected from one or more of acrylate and phthalate polymers or copolymers (e.g. commercially available grades of Eudragit such as RL, RD, RS and NE).

The pharmaceutically acceptable excipients may include one or more diluents, binders, lubricants, glidants, disintegrants, and the like.

Suitable diluents may include one or more of microcrystalline cellulose, di- or tri-basic calcium phosphate, meglumine oxide, crystalline cellulose, powdered cellulose, anhydrous silicic acid, calcium carbonate, calcium sulphate, magnesium silicate, magnesium trisilicate, magnesium aluminium metasilicate (Neusilin), kaolin, starch, starch derivatives, magnesium carbonate, magnesium oxide and co-processed insoluble excipients, and the like.

Suitable disintegrants may include one or more of Veegum (highly refined isomorphous silicate), crospovidone, cellulose, kaolin, crosslinked carboxy methyl cellulose (e.g., AcDiSol), microcrystalline cellulose (e.g., Avicel PH101 & PH102), crosslinked polyvinyl pyrrolidone (e.g., Kollidon CL), and mixtures thereof. Preferred disintegrants among these disintegrants include crosslinked carboxy methyl cellulose (e.g., AcDiSol), microcrystalline cellulose (e.g., Avicel PH101 & PH102), crosslinked polyvinyl pyrrolidone (e.g., Kollidon CL), and the like.

Suitable binders may include one or more of hydroxyethyl cellulose, hydroxypropyl cellulose, hydroxypropyl methylcellulose, carbomers, dextrin, ethyl cellulose, methylcellulose, shellac, zein, gelatin, polymethacrylates, polyvinyl pyrrolidone, pregelatinized starch, sodium alginate, gums, synthetic resins and the like.

Suitable lubricants may include one or more of magnesium stearate, stearic acid, palmitic acid, calcium stearate, zinc stearate, sodium stearyl fumarate, glyceryl behenate, talc and the like.

The orally disintegrating pharmaceutical composition of the present invention can be prepared by any suitable method known in the art such as direct compression, dry or wet granulation, fluidized bed granulation, melt extrusion, melt granulation, spray coating, freeze drying, spray drying and solution evaporation.

In an embodiment, the process of preparing a stable orally disintegrating pharmaceutical composition of lansoprazole or pharmaceutically acceptable salts thereof may include the steps of: (a) layering an inert core with an aqueous suspension comprising lansoprazole or pharmaceutically acceptable salts thereof with one or more pharmaceutically acceptable excipients and an alkalizing agent; and b) coating the drug loaded inert core with one or more enteric polymers and/or pharmaceutically acceptable controlled release polymers.

In a further embodiment, the process of preparing a stable orally disintegrating pharmaceutical composition of lansoprazole or pharmaceutically acceptable salts thereof, may include the steps of (a) mixing lansoprazole or pharmaceutically acceptable salt thereof with one or more alkalizing agents and one or more pharmaceutically acceptable excipients to form an active core; (b) coating the active core with a barrier coating layer; and c) coating the barrier coated core with one or more enteric polymers and/or pharmaceutically acceptable controlled release polymers.

The present invention further provides a method of treating gastroesophageal reflux disease or a symptom thereof in a subject in need thereof, the method comprising administering an orally disintegrating pharmaceutical composition of to the present invention.

In the context of the present invention, “Bioequivalency” is determined by a 90% Confidence Interval (CI) of between 0.80 and 1.25 for both Cₘₐₓ and AUC under USFDA regulatory guidelines, or a 90% Cl for AUC of between 0.80 to 1.25 and a 90% Cl for Cmax of between 0.70 to 1.43 under the European regulatory guidelines (EMEA).

The term “confidence interval, (CI)” as used herein refers to the plain meaning known to one of ordinary skill in the art. The confidence interval refers to a statistical range with a specified probability that a given parameter lies within the range.

The term “covariance, (CV)” as used herein refers to the plain meaning known to one of ordinary skill in the art. It is a statistical measure of the variance of two random variables that are observed or measured in the same mean time period. This measure is equal to the product of the deviations of corresponding values of the two variables from their respective means.

The bioequivalence studies were carried out between Prevacid® (reference) and compositions of the invention (test) in fasted state as well as fed state. The study was monitored in terms of Cₘₐₓ, AUC, Tₘₐₓ achieved with the test products and the reference product (Prevacid®).

At 90% confidence interval; area under the concentration time curve (AUC₀₋ₜ and/or AUC_{0-inf}) and maximum plasma concentration (Cₘₐₓ) values of composition of the invention lies between 0.70 and 1.70 as compared to that obtained by marketed orally disintegrating formulation of lansoprazole marketed under the trade name Prevacid®.

The relative bioavailability study of orally disintegrating composition of lansoprazole of the invention and lansoprazole formulation marketed under the trade name Prevacid® as demonstrated in Tables 5 and 6 concludes that the formulation explored in the present invention provides equivalent extent of absorption compared to that demonstrated by marketed formulation Prevacid®. In addition, the composition of the invention was found to provide patient compliance in terms of oral comfort.

The invention is further illustrated by the following examples which are provided to be exemplary of the invention and do not limit the scope of the invention. While the present invention has been described in terms of its specific embodiments, certain modifications and equivalents will be apparent to those skilled in the art and are intended to be included within the scope of the present invention.

### Example 1

TABLE 1

| | Mg/Tab | |
|---|---|---|
| Ingredients | 30 mg | 15 mg |
| Stage 1: Drug Loading | | |
| Microcrystalline Cellulose Spheres | 85.000 | 42.500 |
| (Celphere CP203) | | |
| Lansoprazole | 30.000 | 15.000 |
| Light Magnesium Carbonate Powder | 12.000 | 6.000 |
| Hypromellose 3 cps | 6.000 | 3.000 |
| Hypromellose 15 cps | 9.000 | 4.500 |
| Sodium Starch Glycolate | 3.000 | 1.500 |
| Talc | 5.000 | 2.500 |
| Water | Q.S | Q.S |
| Total - Drug Pellets | 150.000 | 75.000 |
| Stage 2: Barrier Coating | | |
| Lansoprazole Drug Pellets | 150.000 | 75.000 |
| Magnesium Carbonate Light Powder | 1.980 | 0.990 |
| Hypromellose 6 cps | 9.100 | 4.550 |
| Mannitol 25C | 6.960 | 3.480 |
| Starch | 3.480 | 1.740 |
| Talc | 3.480 | 1.740 |
| Water | Q.S | Q.S |
| Total - Barrier Coated Pellets | 175.000 | 87.500 |
| Stage 3: Enteric Coating | | |
| Lansoprazole Barrier Coated Pellets | 175.000 | 87.500 |
| Methacrylic Acid Copolymer Dispersion | 112.471 | 56.236 |
| (Eudragit L30D-55) | | |
| Triethyl Citrate | 20.245 | 10.123 |
| PEG 6000 | 2.024 | 1.012 |
| Glyceryl Monostearate | 5.624 | 2.812 |
| Polysorbate 80 | 2.812 | 1.406 |
| Water | Q.S | Q.S |
| Total - Enteric coated Pellets | 318.200 | 159.100 |
| Stage 4: Finishing Coating | | |
| Lansoprazole Enteric Coated Pellets | 318.200 | 159.100 |
| Mannitol | 15.800 | 7.900 |
| Water | Q.S | Q.S |
| Total - PEG coated pellets for Compression | 334.000 | 167.00 |
| Stage 5: Blending | | |
| Lansoprazole Finished Coated Pellets | 334.000 | 167.000 |
| Talc | 1.000 | 0.500 |
| Total - Blended Pellets | 335.000 | 167.500 |
| Stage 6: Lubrication and Compression | | |
| Lansoprazole Finished Coated Pellets | 335.000 | 167.500 |
| Mannitol DC 400 | 251.440 | 125.720 |
| Microcrystalline Cellulose (Ceolus KG-802) | 71.840 | 35.920 |
| HD Silicified Microcrystalline Cellulose (Prosolve | 35.920 | 17.960 |
| HD 90) | | |
| Crospovidone (Polyplasdone XL 10) | 58.500 | 29.250 |
| Colloidal Silicon Dioxide (Aerosil 200) | 7.800 | 3.900 |
| Aspartame | 5.850 | 2.925 |
| Flavour Strawberry 052311 AP0551 | 5.850 | 2.925 |
| Magnesium Stearate | 7.800 | 3.900 |
| Total - Lansoprazole DR ODT | 780.000 | 390.000 |

### Procedure:

Microcrystalline cellulose spheres (Celpheres CP 203) were loaded in wurster. These spheres were coated with a dispersion of HPMC 3cps, HPMC 15cps, Magnesium Carbonate light, Lansoprazole, Sodium Starch Glycolate and Talc in Purified water and the resulting pellets were dried. These drug loaded pellets were then coated with a barrier coating solution containing HPMC 6cps, Magnesium Carbonate light, Mannitol, Starch and Talc in Purified water and dried. These barrier coated pellets were then coated with enteric coating dispersion containing Methacrylic Acid Copolymer Dispersion [Eudragit L30D55], glyceryl monostearate, triethyl citrate, PEG 6000 and polysorbate 80 in purified water. The resulting enteric coated pellets were dried and sifted through a suitable sieve. These enteric coated pellets were coated with finishing coating solution containing mannitol in water. The resulting finished coated pellets were dried and blended with talc. This blend is mixed with mannitol DC400, microcrystalline cellulose, HD Silicified Microcrystalline Cellulose (prosolve HD90), crospovidone, colloidal silicon dioxide, aspartame and strawberry flavor in suitable blender. The resulting blend is lubricated with magnesium stearate and compressed into tablets using suitable punches.

### Examples 2 and 3

TABLE 2

| | Mg/Tab | | Mg/Tab | |
|---|---|---|---|---|
| | (Example 2) | | (Example 3) | |
| Ingredients | 30 mg | 15 mg | 30 mg | 15 mg |
| Stage 1: Drug Loading | | | | |
| Microcrystalline Cellulose Spheres | 73.000 | 36.500 | 73.000 | 36.500 |
| (Celphere CP203) | | | | |
| Microcrystalline Cellulose Spheres | 12.000 | 6.000 | 12.000 | 6.000 |
| (Celphere CP305) | | | | |
| Lansoprazole | 30.000 | 15.000 | 30.000 | 15.000 |
| Magnesium Carbonate Light | 12.000 | 6.000 | 10.000 | 5.000 |
| Powder | | | | |
| Hypromellose 3 cps | 9.000 | 4.5.000 | 9.000 | 4.5.000 |
| Hypromellose 15 cps | 6.000 | 3.000 | 6.000 | 3.000 |
| Sodium Starch Glycolate | 3.000 | 1.500 | | |
| Corn starch | | | 5.000 | 2.500 |
| Talc | 5.000 | 2.500 | 5.000 | 2.500 |
| Water | Q.S | Q.S | Q.S | Q.S |
| Total - Drug Pellets | 150.000 | 75.000 | 150.000 | 75.000 |
| Stage 2: Barrier Coating | | | | |
| Lansoprazole Drug Pellets | 150.000 | 75.000 | 150.000 | 75.000 |
| Magnesium Carbonate Light | 1.980 | 0.990 | 1.980 | 0.990 |
| Powder | | | | |
| Hypromellose | 9.100 | 4.550 | 9.100 | 4.550 |
| Mannitol | 6.960 | 3.480 | 6.960 | 3.480 |
| Sodium Starch Glycolate | 3.480 | 1.740 | 3.480 | 1.740 |
| Talc | 3.480 | 1.740 | 3.480 | 1.740 |
| Water | Q.S | Q.S | Q.S | Q.S |
| Total - Barrier Coated Pellets | 175.000 | 87.500 | 175.000 | 87.500 |
| Stage 3: Enteric Coating | | | | |
| Lansoprazole Barrier Coated | 175.000 | 87.500 | 175.000 | 87.500 |
| Pellets | | | | |
| Methacrylic Acid Copolymer | 86.294 | 43.147 | 82.294 | 41.147 |
| Dispersion | | | | |
| Eudragit NE30D | 15.229 | 7.615 | 19.229 | 9.615 |
| Triethyl Citrate | 16.613 | 8.306 | 1.661 | 0.831 |
| PEG 6000 | 1.661 | 0.831 | 16.613 | 8.306 |
| Glyceryl Monostearate | 4.615 | 2.307 | 4.615 | 2.307 |
| Polysorbate 80 | 2.307 | 1.154 | 2.307 | 1.154 |
| Ferric Oxide Red | 0.021 | 0.011 | 0.021 | 0.011 |
| Water | Qs | Qs | Qs | Qs |
| Total - Enteric coated Pellets | 301.740 | 150.870 | 301.740 | 150.870 |
| Stage 4: Finishing Coating | | | | |
| Lansoprazole Enteric Coated | 301.740 | 150.870 | 301.740 | 150.870 |
| Pellets | | | | |
| PEG 6000 | 15.160 | 7.580 | 15.160 | 7.580 |
| Water | Qs | Qs | Qs | Qs |
| for Total - Finished coated | 316.900 | 158.450 | 316.900 | 158.450 |
| pellets Compression | | | | |
| Stage 5: Blending | | | | |
| Lansoprazole Finished Coated | 316.900 | 158.450 | 316.900 | 158.450 |
| Pellets | | | | |
| Talc | 1.100 | 0.550 | 1.100 | 0.550 |
| Total - Blended Pellets | 318.000 | 159.000 | 318.000 | 159.000 |
| Stage 6: Lubrication and | | | | |
| Compression | | | | |
| Lansoprazole Finished Coated | 318.000 | 159.000 | 318.000 | 159.000 |
| Pellets | | | | |
| Mannitol DC 400 | 263.340 | 131.670 | 263.340 | 131.670 |
| Microcrystalline Cellulose | 75.240 | 37.620 | 75.240 | 37.620 |
| HD Silicified Microcrystalline | 37.620 | 18.810 | 37.620 | 18.810 |
| Cellulose | | | | |
| Crospovidone | 58.500 | 29.250 | 58.500 | 29.250 |
| Colloidal Silicon Dioxide | 7.800 | 3.900 | 7.800 | 3.900 |
| Aspartame | 5.850 | 2.925 | 5.850 | 2.925 |
| Flavour Strawberry | 5.850 | 2.925 | 5.850 | 2.925 |
| Magnesium Stearate | 7.800 | 3.900 | 7.800 | 3.900 |
| Total | 780.00 | 390.000 | 780.00 | 390.000 |

### Procedure:

Microcrystalline cellulose spheres (Celpheres CP 203 & Celphere CP305) were loaded in wurster. These spheres were coated with a dispersion of HPMC 3cps, HPMC 15cps, Magnesium Carbonate light, Lansoprazole, Sodium Starch Glycolate, Corn Starch and Talc in Purified water and the resulting pellets were dried. These drug loaded pellets were then coated with a barrier coating solution containing HPMC 6cps, Magnesium Carbonate light, Mannitol, Corn Starch, Sodium Starch Glycolate and Talc in Purified water and dried. These barrier coated pellets were then coated with enteric coating dispersion containing Methacrylic Acid Copolymer Dispersion [Eudragit L30D55], Eudragit NE30D, glyceryl monostearate, triethyl citrate, PEG 6000 and Polysorbate 80 in purified water. The resulting enteric coated pellets were dried and sifted through a suitable sieve. These enteric coated pellets were coated with finishing coating solution containing PEG 6000 in water. The resulting finished coated pellets were dried and blended with talc. This blend is mixed with Mannitol DC400, Microcrystalline Cellulose, HD Silicified Microcrystalline Cellulose (Prosolve HD90); Crospovidone, Colloidal silicon dioxide, Aspartame and Strawberry flavor in suitable blender. The resulting blend is lubricated with Magnesium stearate and compressed into tablets using suitable punches.

### Examples 4 and 5

TABLE 3

| | Mg/Tab | | Mg/Tab | |
|---|---|---|---|---|
| | (Example 4) | | (Example 5) | |
| Ingredients | 30 mg | 15 mg | 30 mg | 15 mg |
| Stage 1: Drug Loading | | | | |
| Microcrystalline Cellulose Spheres | 80.000 | 40.000 | 73.000 | 36.500 |
| (Celphere CP203) | | | | |
| Microcrystalline Cellulose Sphere | | | 12.000 | 6.000 |
| (Celphere CP305) | | | | |
| Lansoprazole | 30.000 | 15.000 | 30.000 | 15.000 |
| Magnesium Carbonate Light | 12.000 | 6.000 | 10.000 | 5.000 |
| Powder | | | | |
| Hypromellose 3 cps | 9.000 | 4.5.000 | 9.000 | 4.5.000 |
| Hypromellose 15 cps | 6.000 | 3.000 | 6.000 | 3.000 |
| Sodium Starch Glycolate | 3.000 | 1.500 | | |
| Corn starch | | | 5.000 | 2.500 |
| Talc | 10.000 | 5.000 | 5.000 | 2.500 |
| Water | Q.S | Q.S | Q.S | Q.S |
| Total - Drug Pellets | 150.000 | 75.000 | 150.000 | 75.000 |
| Stage 2: Barrier Coating | | | | |
| Lansoprazole Drug Pellets | 150.000 | 75.000 | 150.000 | 75.000 |
| Magnesium Carbonate Light | | | 2.000 | 1.000 |
| Powder | | | | |
| Hypromellose | 9.100 | 4.550 | 9.100 | 4.550 |
| Mannitol | 8.940 | 4.470 | 6.940 | 3.470 |
| Sodium Starch Glycolate | 3.480 | 1.740 | 3.480 | 1.740 |
| Talc | 3.480 | 1.740 | 3.480 | 1.740 |
| Water | Q.S | Q.S | Q.S | Q.S |
| Total - Barrier Coated Pellets | 175.000 | 87.500 | 175.000 | 87.500 |
| Stage 3: Enteric Coating | | | | |
| Lansoprazole Barrier Coated | 175.000 | 87.500 | 175.000 | 87.500 |
| Pellets | | | | |
| Methacrylic Acid Copolymer | 86.294 | 43.147 | 82.294 | 41.147 |
| Dispersion | | | | |
| Eudragit NE30D | 15.229 | 7.615 | 19.229 | 9.615 |
| Triethyl Citrate | 18.274 | 9.137 | | |
| PEG 6000 | | | 18.274 | 9.137 |
| Glyceryl Monostearate | 4.615 | 2.307 | 4.615 | 2.307 |
| Polysorbate 80 | 2.307 | 1.154 | 2.307 | 1.154 |
| Ferric Oxide Red | 0.021 | 0.011 | 0.021 | 0.011 |
| Water | Qs | Qs | Qs | Qs |
| Total - Enteric coated Pellets | 301.740 | 150.870 | 301.740 | 150.870 |
| Stage 4: Finishing Coating | | | | |
| Lansoprazole Enteric Coated | 301.740 | 150.870 | 301.740 | 150.870 |
| Pellets | | | | |
| PEG 8000 | 15.160 | 7.580 | 15.160 | 7.580 |
| Water | Qs | Qs | Qs | Qs |
| Total - Finished coated pellets | 316.900 | 158.450 | 316.900 | 158.450 |
| for Compression | | | | |
| Stage 5: Blending | | | | |
| Lansoprazole Finished Coated | 316.900 | 158.450 | 316.900 | 158.450 |
| Pellets | | | | |
| Talc | 1.100 | 0.550 | 1.100 | 0.550 |
| Total - Blended Pellets | 318.000 | 159.000 | 318.000 | 159.000 |
| Stage 6: Lubrication and | | | | |
| Compression | | | | |
| Lansoprazole Finished Coated | 318.000 | 159.000 | 318.000 | 159.000 |
| Pellets | | | | |
| Mannitol DC 400 | 263.340 | 131.670 | 263.340 | 131.670 |
| Microcrystalline Cellulose | 75.240 | 37.620 | 75.240 | 37.620 |
| HD Silicified Microcrystalline | 37.620 | 18.810 | 37.620 | 18.810 |
| Cellulose | | | | |
| Crospovidone | 58.500 | 29.250 | 58.500 | 29.250 |
| Colloidal Silicon Dioxide | 7.800 | 3.900 | 7.800 | 3.900 |
| Aspartame | 5.850 | 2.925 | 5.850 | 2.925 |
| Flavour Strawberry | 5.850 | 2.925 | 5.850 | 2.925 |
| Magnesium Stearate | 7.800 | 3.900 | 7.800 | 3.900 |
| Total | 780.00 | 390.000 | 780.00 | 390.000 |

### Procedure:

Microcrystalline cellulose spheres (Celpheres CP 203 & Celphere CP305) were loaded in wurster. These spheres were coated with a dispersion of HPMC 3cps, HPMC 15cps, Magnesium Carbonate light, Lansoprazole, Sodium Starch Glycolate, Corn Starch and Talc in Purified water and the resulting pellets were dried. These drug loaded pellets were then coated with a barrier coating solution containing HPMC 6cps, Magnesium Carbonate light, Mannitol, Corn Starch, Sodium Starch Glycolate and Talc in Purified water and dried. These barrier coated pellets were then coated with enteric coating dispersion containing Methacrylic Acid Copolymer Dispersion [Eudragit L30D55[, Eudragit NE30D, glyceryl monostearate, triethyl citrate, PEG 6000 and Polysorbate 80 in purified water. The resulting enteric coated pellets were dried and sifted through a suitable sieve. These enteric coated pellets were coated with finishing coating solution containing PEG 8000 in water. The resulting finished coated pellets were dried and blended with talc. This blend is mixed with Mannitol DC400, Microcrystalline Cellulose, HD Silicified Microcrystalline Cellulose (Prosolve HD90), Crospovidone, Colloidal silicon dioxide, Aspartame and Strawberry flavor in suitable blender. The resulting blend is lubricated with Magnesium stearate and compressed into tablets using suitable punches.

### Example 6

### Stability Data

Stability data of the composition of invention was carried out using storage condition of 40° C. and 75% relative humidity.

TABLE 4

| | | | | | | Single | |
|---|---|---|---|---|---|---|---|
| | | | | | | Max. | |
| Batch | | | Known Impurities | | | Unknown | Total |
| No. | Condition | Pack | A | B | C | Impurity | Impurity |
| | | Limit | 0.25 | 0.25 | 0.25 | 0.20 | ND |
| I | Initial | Alu- | 0.08 | 0.02 | 0.10 | ND | 0.21 |
| | 40° C./ | Alu- | 0.13 | 0.02 | 0.04 | 0.18 | 0.70 |
| | 75% RH-3 | Blister | | | | | |
| | Months | | | | | | |
| II | Initial | Alu- | 0.08 | 0.03 | 0.02 | 0.07 | 0.26 |
| | 40° C./ | Alu- | 0.11 | 0.03 | 0.06 | 0.16 | 0.68 |
| | 75% RH-3 | Blister | | | | | |
| | Months | | | | | | |

### Example 7

### In-Vivo Pharmacokinetic Parameters

Bioavailability study of the composition of the invention was carried out on healthy volunteers taking marketed formulation Prevacid® as the reference, the results of which are represented in Tables 5 and 6.

### A) Fasting Data

TABLE 5

| | Ratio | | | |
|---|---|---|---|---|
| Dependent | [% Ref] | CI_90_Lower | CI_90_Upper | Power |
| Ln (AUCINF_obs) | 96.78 | 84 | 112 | 0.8 |
| Ln (AUClast) | 96.97 | 84 | 112 | 0.8 |
| Ln (Cmax) | 97.34 | 82 | 116 | 0.7 |

### B) Fed Data

TABLE 6

| | Ratio | | | |
|---|---|---|---|---|
| Dependent | [% Ref] | CI_90_Lower | CI_90_Upper | Power |
| Ln (AUCINF_obs) | 112.60 | 102 | 125 | 0.9 |
| Ln (AUClast) | 112.58 | 101 | 125 | 0.9 |
| Ln (Cmax) | 100.74 | 89 | 114 | 0.9 |

While the invention has been described in terms of its specific embodiments, certain modifications and equivalents will be apparent to those skilled in the art and are intended to be included within the scope of the invention.

We claim:

1. An orally disintegrating pharmaceutical composition comprising enteric coated granules of lansoprazole or pharmaceutically acceptable salts thereof, wherein the enteric coated granules have an average particle diameter of more than 400 μm.

2. The orally disintegrating pharmaceutical composition as claimed in claim 1, wherein the enteric coated granules have an average particle diameter in the range of about 400 μm to about 750 μm.

3. The orally disintegrating pharmaceutical composition as claimed in claim 1, wherein the enteric coated granules are coated with an enteric coating layer comprising one or more enteric polymer/s and optionally, one or more pharmaceutically acceptable controlled release polymers.

4. The orally disintegrating pharmaceutical composition as claimed in claim 3, wherein the enteric polymer comprises one or more of hydroxypropylmethyl cellulose phthalate, cellulose acetate phthalate, polyvinyl acetate phthalate, methyl cellulose phthalate, copolymerized methacrylic acid/methacrylic acid methyl esters and methacrylate copolymer.

5. The orally disintegrating pharmaceutical composition as claimed in claim 3, wherein the pharmaceutically acceptable controlled release polymer comprises one or more of hydrophilic and hydrophobic polymer/s.

6. The orally disintegrating pharmaceutical composition as claimed in claim 5, wherein the pharmaceutically acceptable controlled release polymer is an acrylate copolymer.

7. The orally disintegrating pharmaceutical composition as claimed in claim 3, wherein the amount of enteric coating layer comprises from about 35% to about 55% based on the total weight of the enteric coated granules in the composition.

8. The orally disintegrating pharmaceutical composition as claimed in claim 1, wherein the enteric coated granules comprise one or more over-coating layers.

9. The orally disintegrating pharmaceutical composition as claimed in claim 8, wherein the over-coating layer comprises one or more sugar alcohols.

10. The orally disintegrating pharmaceutical composition as claimed in claim 1, wherein the enteric coated granules comprise:
(a) a drug core comprising lansoprazole or pharmaceutically acceptable salts thereof, one or more pharmaceutically acceptable excipients, and optionally one or more alkalizing agents;
(b) an optional barrier coating layer over the drug core comprising one or more pharmaceutically acceptable excipients, and optionally one or more alkalizing agents; and
(c) an outer enteric coating layer comprising one or more enteric polymers, and optionally one or more pharmaceutically acceptable controlled release polymers.

11. The orally disintegrating pharmaceutical composition as claimed in claim 1, wherein the composition further comprises one or more pharmaceutically acceptable excipients comprising one or more diluents, disintegrants, binders, lubricants and/or glidants.

12. The orally disintegrating pharmaceutical composition as claimed in claim 1, wherein the composition is provided in the form of a tablet, a capsule, granules, pellets, caplets, minitablets, a capsule filled with minitablets and/or pellets, a multi-layer tablet, granules for suspension, or granules and powder filled in a sachet.

13. The orally disintegrating pharmaceutical composition as claimed in claim 1, wherein the composition exhibits no significant difference in rate and/or extent of absorption of lansoprazole or pharmaceutically acceptable salts thereof as compared to orally disintegrating formulation of lansoprazole commercially marketed under the trade name Prevacid®.

14. The orally disintegrating pharmaceutical composition as claimed in claim 1, wherein the composition disintegrates in less than 30 seconds in water at 37° C.

15. A stable orally disintegrating pharmaceutical composition comprising enteric coated granules of lansoprazole or pharmaceutically acceptable salts thereof, wherein the granules have an average particle diameter of more than 400 μm, wherein the composition retains at least 80% of the potency of lansoprazole or pharmaceutically acceptable salts thereof in the pharmaceutical composition after storage for three months at 40° C. and 75% relative humidity.

16. An orally disintegrating pharmaceutical composition comprising enteric coated granules, wherein the enteric coated granules comprise:
(a) about 35% to about 65% by weight of a drug core comprising lansoprazole or pharmaceutically acceptable salts thereof, one or more pharmaceutically acceptable excipients, and optionally one or more alkalizing agents;
(b) about 5% to about 15% by weight of an optional barrier coating layer over the drug core comprising one or more pharmaceutically acceptable excipients, and optionally one or more alkalizing agents;
(c) about 30% to about 60% by weight of an enteric coating layer over the drug core or barrier coating layer comprising one or more enteric polymers, and optionally one or more pharmaceutically acceptable controlled release polymers; and
(d) about 2% to about 8% by weight of an over-coating layer over the enteric coating layer comprising one or more pharmaceutically acceptable excipients,
wherein the enteric coated granules have an average particle diameter of more than 400 μm.

17. The orally disintegrating pharmaceutical composition as claimed in claim 16, wherein the core comprises about 1% to about 15% by weight of an alkalizing agent.

18. The orally disintegrating pharmaceutical composition as claimed in claim 16, wherein the barrier coating layer comprises about 0.01% to about 2% by weight of the alkalizing agent.

19. The orally disintegrating pharmaceutical composition as claimed in claim 16, wherein the enteric coating layer comprises about 15% to about 30% by weight of one or more enteric polymer/s and about 0.5% to about 5% by weight of one or more pharmaceutically acceptable controlled release polymer/s.

20. A process for the preparation of an orally disintegrating pharmaceutical composition of lansoprazole or pharmaceutically acceptable salts thereof, the process comprising providing enteric coated granules comprising lansoprazole or pharmaceutically acceptable salts thereof, one or more pharmaceutically acceptable excipients, and optionally one or more alkalizing agents, wherein the enteric coated granules have an average particle diameter of more than 400 μm; forming a mixture by mixing the enteric coated granules with one or more pharmaceutically acceptable excipients; and forming the mixture into a pharmaceutical dosage form.

21. The process as claimed in claim 20, wherein the pharmaceutical dosage form is a tablet, a capsule, granules, pellets, caplets, minitablets, a capsule filled with minitablets and/or pellets, a multi-layer tablet, granules for suspension, or granules and powder filled in a sachet.

22. A method of treating gastroesophageal reflux disease or a symptom thereof in a subject in need thereof, the method comprising administering an orally disintegrating pharmaceutical composition comprising enteric coated granules of lansoprazole or pharmaceutically acceptable salts thereof, wherein the enteric coated granules have an average particle diameter of more than 400 μm.
